(19) United States
(12) Patent Application Publication
(10) Pub. No.: US 2025/0182883 A1
(43) Pub. Date: Jun. 5, 2025

(54) SYSTEMS AND METHODS FOR IMAGING AND ANATOMICAL MODELING

(71) Applicant: Shifamed Holdings, LLC, Campbell, CA (US)

(72) Inventors: Claudio Argento, Felton, CA (US); Amr Salahieh, Saratoga, CA (US); Alejandro Rendon Sanchez, Woodside, CA (US)

(21) Appl. No.: 18/833,350

(22) PCT Filed: Jan. 30, 2023
(86) PCT No.: PCT/US2023/061573

§ 371 Date: Jul. 25, 2024

Related U.S. Application Data
(60) Provisional application No. 63/267,285, filed on Jan. 28, 2022.
(60) Provisional application No. 63/365,743, filed on Jun. 2, 2022.
(60) Provisional application No. 63/386,850, filed on Dec. 9, 2022.

(51) Int. Cl. G16H 30/40; G16H 50/50

(52) U.S. Cl. CPC G16H 30/40; G16H 50/50

(57) ABSTRACT

Systems and methods are provided for imaging a target anatomy of a patient. The systems and methods can include generating and updating a 3D model of the target anatomy. In some embodiments, the target anatomy can be a heart of the patient. The 3D model can be updated with image slices obtained with a catheter based imaging system, such as a TEE imaging system. In some examples, the images can be applied to a classifier to determine if the present view corresponds to a user-selected or desired view. The classifier score can be used to provide instructions to a control system to move the imaging system until the desired view is obtained.

### CLAIM OF PRIORITY

This application claims the benefit of priority to U.S. Application No. 63/267,285, filed Jan. 28, 2022, U.S. Application No. 63/365,743, filed Jun. 2, 2022, and U.S. Application No. 63/386,850, filed Dec. 9, 2022.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The inventions relate, in general, to medical imaging and modeling, and methods for their use. Various aspects of the inventions relate to use of robotic tools for such imaging and modeling.

### BACKGROUND

Medical imaging has advanced significantly in recent years with the introduction of new imaging modalities and vast improvements in computing power. Common examples include ultrasound for imaging anatomical bodies, such as transesophageal echocardiography (TEE), intravascular ultrasound (IVUS), and other imaging modalities as known in the art. Clinicians widely use imaging tools for diagnosis, assessment, treatment planning, intraoperative guidance, and more. Composite images are often used to create an anatomical map, such as with cardiac mapping systems.

However, existing imaging systems have significant limitations even with the recent advances. Echocardiography, for example, produces images which require a high degree of skill to interpret. Moreover, even skilled clinicians typically take considerable time to position the probe to optimize the images produced. Although the images can be in real-time, they are fixed inasmuch as the images are taken from a single location. The clinician must go through the tedious and difficult process of repositioning the probe to image different anatomical structures or even different angles of the same structure. The image rendered on the screen only shows image information from a single particular view, thus it does not display information about structures outside the field of view. Furthermore, these images are typically degraded by noise, shadows, etc.

Many procedures require the presence of these highly skilled echocardiographers, which necessitates tight coordination and communication between the interventionalist and echocardiographer. This can lead to increased crowding, noise, cost, and delays during procedures due to lack of availability of electrocardiologists.

Recently 3D modeling and imaging systems have been introduced but still have several limitations. Cardiac mapping systems can model the entire heart but only identify temporal and spatial distributions of electrical potentials. They do not show other information like, for example, an anatomical model. More recently technologies have been developed to display true three-dimensional models based on medical imaging, but these technologies likewise suffer from many limitations including poor image quality, incomplete image information, slow processing, and more. These technologies also increase the workload of the already over-burdened clinical team.

There remains a need for a system to address the above needs and more. There remains a need for improved imaging and modeling tools and methods. There remains a need for imaging tools that enable greater flexibility and provide more information for clinicians. There remains a need for high order imaging information that can be accessed by less experienced clinicians. These and other needs are met by the present inventions.

### SUMMARY

A method of automatically building and/or updating a cardiovascular model, comprising obtaining first image data from a first location in a patient, the first image data including information related to at least one anatomical structure, obtaining second image data from a second location in a patient, the second image data including information related to the at least one anatomical structure and generating a representation of the at least one anatomical structure based on the first and second image data.

In some embodiments, the generating includes building a representation of a 3D anatomical model.

In one example, the method further comprises obtaining third image data relating to the at least one anatomical structure, determining a correspondence between the third image data and the 3D anatomical model, identifying a discrepancy between the at least one anatomical structure in the third image data and the associated at least one structure in the 3D anatomical model, and updating the 3D anatomical model based on the discrepancy.

An imaging system is provided for use in modelling an anatomical structure of a patient, comprising a catheter sized and shaped for percutaneous insertion into the patient, an imaging probe having a field of view, coupled to the catheter near a distal end thereof, a drive mechanism coupled to the catheter and/or the imaging probe, configured to translate and/or rotate the imaging probe, and a processor operatively coupled to the imaging probe and the drive mechanism, the processor configured to transmit and receive signals with the drive mechanism and the imaging probe, to—control the drive mechanism to place the imaging probe at a first and a second position within the patient, and control the imaging probe to generate first image data and second image data related to respective first and second fields of view therefrom, wherein the processor is configured to generate and/or update a model of the anatomical structure considering the first and second image data.

A method of automatically building and/or updating a cardiovascular model is provided, comprising generating an initial 3D model of at least one anatomical structure based on a standard distribution of corresponding anatomical structures, obtaining first image data from a first location in a patient, the first image data including information related to a portion of the at least one anatomical structure, obtaining second image data from a second location in a patient, the second image data including information related to a different portion of the at least one anatomical structure, and generating a modified 3D model of the at least one anatomical structure based on the first and second image data and the initial 3D model.

In some embodiments, the at least one anatomical model comprises a heart.

In one implementation, obtaining first and second image data comprises obtaining first TEE image data and second TEE image data.

In some embodiments, the method further comprises updating the standard distribution of corresponding anatomical structures with the first and second image data.

In one embodiment, the method further includes a model of a selected therapeutic procedure or tool in the modified 3D model.

In some examples, generating the initial 3D model further comprises generating an initial 3D physics model of the at least one anatomical structure.

A non-transitory computing device readable medium is provided having instructions stored thereon that are executable by a processor to cause a computing device to perform the method of obtaining first image data from a first location in a patient, the first image data including information related to at least one anatomical structure, obtaining second image data from a second location in a patient, the second image data including information related to the at least one anatomical structure, and generating a representation of the at least one anatomical structure based on the first and second image data.

An imaging system is provided for use in modelling an anatomical structure of a patient, comprising a robotically-controlled drive mechanism configured to be coupled to and drive an imaging probe, and a processor operatively coupled to the imaging probe and the drive mechanism, the processor configured to transmit and receive signals with the drive mechanism and the imaging probe, to—control the drive mechanism to place the imaging probe at a first and a second position within the patient, and collect image data from imaging probe at the first and second positions, wherein the processor is configured to generate and/or update a model of the anatomical structure considering the first and second image data.

A method of automatically building and/or updating a cardiovascular model is provided, comprising obtaining in a non-transitory computing device readable medium first image data from a first location in a patient, the first image data including information related to at least one anatomical structure, obtaining in the non-transitory computing device readable medium second image data from a second location in a patient, the second image data including information related to the at least one anatomical structure, and executing instructions stored in the non-transitory computing device readable medium with a processor to cause the computing device to generate a representation of the at least one anatomical structure based on the first and second image data.

A method of building and/or updating a cardiovascular model is provided, comprising moving an imaging probe to a first location with a robotic arm of a robotic positioning system, obtaining first image data from the first location, the first image data including information related to at least one anatomical structure, moving the imaging probe to a second location with the robotic arm, obtaining second image data from the second location, the second image data including information related to the at least one anatomical structure, and updating a representation of the at least one anatomical structure based on the first and second image data.

In some embodiments, the method further comprises moving the imaging probe to a third location with the robotic arm, obtaining third image data relating to the at least one anatomical structure, determining a correspondence between the third image data and the 3D anatomical model, identifying a first discrepancy between the at least one anatomical structure in the third image data and the associated at least one structure in the 3D anatomical model, and moving the imaging probe with the robotic arm to a fourth location calculated from the first discrepancy.

In some embodiments, the method further comprises obtaining fourth image data relating to the at least one anatomical structure.

In some examples, the method further comprises identifying a second discrepancy between the at least one anatomical structure in the third image data and the at least one anatomical structure in the fourth image data.

In some examples, the method includes moving the imaging probe with the robotic arm to a fifth location calculated from the second discrepancy.

An imaging system for use in modelling an anatomical structure of a patient is provided, comprising a catheter sized and shaped for percutaneous insertion into the patient, an imaging probe having a field of view, coupled to the catheter near a distal end thereof, a robotic arm coupled to the catheter and/or the imaging probe, configured to translate and/or rotate the imaging probe, a mouthpiece configured to be worn by the patient, the mouthpiece being configured to receive the catheter, a processor operatively coupled to the imaging probe and the drive mechanism, the processor configured to transmit and receive signals with the drive mechanism and the imaging probe, to—control the robotic arm to place the imaging probe at a first and a second position within the patient, and control the imaging probe to generate first image data and second image data related to respective first and second fields of view therefrom, wherein the processor is configured to generate and/or update a model of the anatomical structure considering the first and second image data.

In some embodiments, the system includes one or more sensors disposed on or within the mouthpiece and being configured to measure one of an axial movement of the catheter or a rotation of the catheter with respect to the mouthpiece.

In one example, the system includes a rigid attachment between the mouthpiece and the robotic arm.

In another embodiment, the system includes a rigid attachment between the mouthpiece and a handle of the catheter. In some embodiments, the rigid attachment comprises a rack and pinion system. In another example, the rigid attachment is configured to prevent excessive forces from being translated by the robotic arm to the patient or the mouthpiece.

An imaging system for use in modelling an anatomical structure of a patient is provided, comprising a robotically-controlled drive mechanism configured to be coupled to and drive an imaging probe, and a processor operatively coupled to the imaging probe and the drive mechanism, the processor configured to transmit and receive signals with the drive mechanism and the imaging probe, to—control the drive mechanism to place the imaging probe at a first and a second position within the patient, and collect image data from imaging probe at the first and second positions, wherein the processor is configured to generate and/or update a model of the anatomical structure considering the first and second image data.

A robotic system for use in control of an imaging catheter, the robotic system comprising a base (e.g., comprising lockable wheels) adapted to be repositionable along a floor within an operating room, an arm movably coupled to the base, the arm comprising an interface for receiving a middle portion of an elongate shaft of the imaging catheter, a cradle sized and shaped for receiving an imaging catheter handle therein, the cradle comprising one or more actuators positioned to interface with one or more knobs of the imaging catheter handle when the imaging handle is secured within the cradle, wherein the cradle is adapted for translation and/or rotation; and a controller operatively coupled to the arm and the cradle, the controller programmed to cause movement of the 1) arm, 2) cradle, and/or 3) cradle actuators to adjust a position of the imaging catheter or a configuration of a knob thereof.

In some embodiments, the robotic system further comprises an imaging console adapted to receive and process imaging data from the imaging catheter.

In some implementations, the controller is operatively coupled to the imaging console and is further programmed to cause the movement (in d. of claim 1) considering the processed imaging data.

A view-based imaging system is provided, comprising a catheter, an imaging element disposed on the catheter, a console operatively coupled to the catheter and the imaging element, the console being configured to display image information from the catheter and include input controls for selecting a desired view within a patient, a control system configured to manipulate a position and/or orientation of the catheter and/or imaging element, the control system being configured to move the catheter and/or imaging element within the patient such that the imaging element obtains the desired view, and one or more processors and memory coupled to the one or more processors, the memory being configured to store computer-program instructions, that, when executed by the one or more processors automatically classifies a present view of the imaging element and provides instructions to the control system to move the imaging element to a location that optimizes the desired view.

In some embodiments, the computer-program instructions are further configured to apply a score to the present view.

In another embodiment, the computer-program instructions are further configured to provide instructions to the control system to move the imaging element until the score for the present view is above a target threshold.

In some embodiments, the computer-program instructions are further configured to provide instructions to the control system to move the imaging element until the score for the present view is maximized.

A system is provided, comprising one or more processors, memory coupled to the one or more processors, the memory configured to store computer-program instructions, that, when executed by the one or more processors, implement a computer-implemented method, the computer-implemented method comprising receiving input controls from a user selecting a desired view of a patient's anatomy, obtaining a first image from an imaging element of a catheter positioned at a first location within the patient, applying the first image to a classifier to obtain a score that indicates if the first image corresponds to the desired view, if the score is above a threshold, indicating to the user that the first image corresponds to the desired view, if the score is below the threshold, providing instructions to move the imaging element of the catheter to a second location, and applying the second image to the classifier to obtain a new score that indicates if the second image corresponds to the desired view.

In some embodiments, the system includes repeating providing instructions to move the imaging element to subsequent locations and applying images from the subsequent locations until the classifier returns a new score indicating that the image corresponds to the desired view.

A method is provided, comprising receiving input controls from a user selecting a desired view of a patient's anatomy, obtaining a first image from an imaging element of a catheter positioned at a first location within the patient, applying the first image to a classifier to obtain a score that indicates if the first image corresponds to the desired view, if the score is above a threshold, indicating to the user that the first image corresponds to the desired view, if the score is below the threshold providing instructions to move the imaging element of the catheter to a second location, and applying the second image to the classifier to obtain a new score that indicates if the second image corresponds to the desired view.

In some embodiments, the method further comprises repeating providing instructions to move the imaging element to subsequent locations and applying images from the subsequent locations until the classifier returns a new score indicating that the image corresponds to the desired view.

### BRIEF DESCRIPTION OF THE FIGURES

FIG. 1 is an exemplary view of a heart and circulatory system of a patient.

FIG. 2 is a schematic view of the system in accordance with the inventions imaging the heart of FIG. 1.

FIG. 3 is one embodiment of a system according to one embodiment of the invention.

FIGS. 4A-4B illustrate one embodiment of a system and method for generating and updating a 3D model of a patient's heart.

FIG. 5 is a flowchart showing one embodiment of development and use of a 3D model of a patient's heart during a procedure.

FIG. 6 is a flowchart of a top level algorithm for generating and displaying a 3D model of a patient's heart.

FIGS. 7A-7B are schematic views of a system in accordance with the inventions imaging the heart.

FIG. 8 illustrates one embodiment of a system and method for generating and updating a 3D model of a patient's heart.

FIG. 9 is a schematic of one or more methods of the present disclosure.

FIGS. 10-11 illustrate one embodiment of a system including a robot.

FIGS. 12A-12D illustrate one embodiment of a system for robotically controlling a position of a catheter and probe.

FIGS. 13A-13D illustrate another embodiment of a system for robotically controlling a position of a catheter and probe.

FIGS. 14A-14I illustrate a method of robotically positioning a probe based on confidence levels of a view of the probe with respect to a 3D model.

### DETAILED DESCRIPTION

Reference will now be made in detail to the preferred embodiments of the invention, examples of which are illustrated in the accompanying drawings. While the invention will be described in conjunction with the preferred embodiments, it will be understood that they are not intended to limit the invention to those embodiments. On the contrary, the invention is intended to cover alternatives, modifications and equivalents, which may be included within the spirit and scope of the invention as defined by the appended claims.

For convenience in explanation and accurate definition in the appended claims, the terms “up” or “upper”, “down” or “lower”, “inside” and “outside” are used to describe features of the present invention with reference to the positions of such features as displayed in the figures.

In many respects the modifications of the various figures resemble those of preceding modifications and the same reference numerals followed by subscripts “a”, “b”, “c”, and “d” designate corresponding parts.

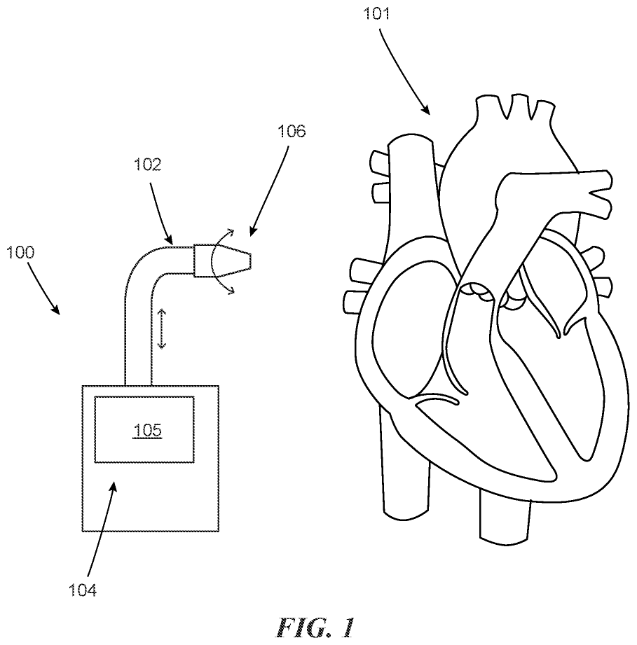

In one embodiment shown in FIG. 1, a system 100 is shown for imaging a part of an anatomical structure 101 (e.g., a heart). The system 100 can include, for example, a catheter 102 and a console 104 having an optional display 105. In various embodiments, the imaging probe and console may be similar in certain respects to transesophageal echocardiography (TEE), transthoracic, and other medical imaging systems. The exemplary system includes a console 104 positioned outside the body and a probe 106 positioned on the end of a catheter 102 for positioning inside the body. The catheter and probe can be configured for esophageal and/or percutaneous insertion, for example.

The probe may be one of a variety of imaging modalities. Examples include an ultrasound transducer. Such imaging probes may be known by one of skill in the art from the description herein including, but not limited to, probes used for transthoracic and/or TEE (e.g., 2D spatial+1D time=3D and 3D spatial+1D time=4D). In various embodiments, the probe is a mini-TEE probe. In various embodiments, the probe is miniaturized by including only the necessary number of signal lines and imaging elements. In some embodiments, the system can include a plurality of imaging modalities and can be configured to multiplex through the different imaging modalities to acquire the necessary images (e.g., transthoracic probes and/or other imaging technologies such as fluoroscopy, CT, etc.).

The probe 106 can be electrically connected to the console 104. Processing of the data collected by the probe may be accomplished via electronics and software in the console. The console may include, for example, various processors, power supplies, memory, firmware, and software configured to receive, store, and process data collected by the probe 106. Various types of probes may be used as would be understood by one of skilled in the art.

In various embodiments, the catheter device may be controllable and automated, such as by robotic control. The catheter, such as the distal end of the catheter, may be advanced and retracted axially from the console, and the probe may be steerable in multiple degrees of freedom, as indicated by the arrows in FIG. 1. For example, the probe and/or catheter may be coupled or attached to a robotic surgical system or robotic positioning system, that may include one or more robotic arms. In various embodiments, the system is configured to store and interpret data taken by the probe in multiple locations in the time domain. In various embodiments, the system uses the interpreted data to generate information for a clinician. For example, the system may construct a 3D anatomical model based on image data taken from multiple locations. In another example, the system generates image data based on composite data from multiple locations.

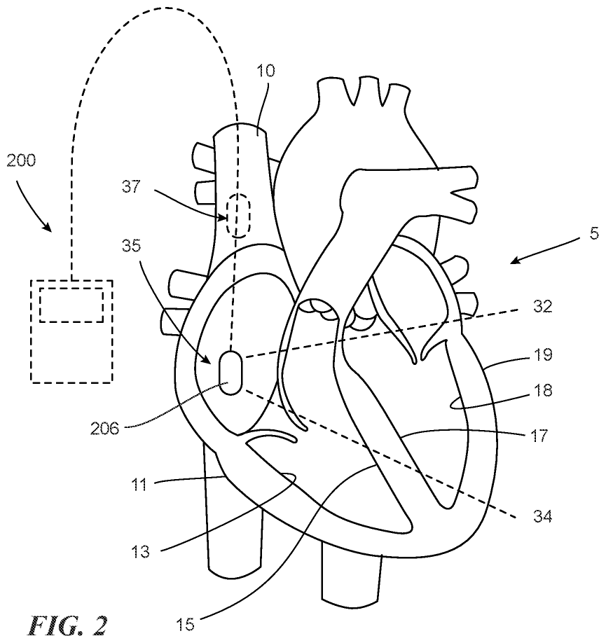

Referring to FIG. 2, the probe 206 can be positioned in the superior vena cava 10 of a patient facing the target anatomy (e.g., heart) 5. With a conventional system the clinician may be presented with images of only one or two anatomical structures, such as structures 11 and 13, in this case the myocardial wall and right ventricular wall, respectively (high order information). Other structures such as the opposite ventricular wall 15, left ventricular walls 17 and 18, and myocardial wall 19 are not displayed with conventional systems because they are too far for sufficient resolution (low order information). By contrast, with the inventive system of the present disclosure, any structures within the field of view (illustrated by dotted lines 32 and 34) may be imaged. This is accomplished as described in more detail below.

The system 200 is designed to obtain image data in multiple locations. In a single location the imaging data for a target structure may not capture the entire structure. When moved to another location, however, the system combines the imaging data from multiple locations with other information including, but not limited to, positional and temporal data.

As shown in exemplary FIG. 2, the probe may take an image at location 35 and then capture new image data at another location 37. The image data from both locations can stored in memory (e.g., either directly on the probe or catheter of the system, or remotely such as in the console or in a remote server) and/or processed and combined.

The system may take into account positional information. For example, the system may recognize that location 35 is in the superior vena cava 10. The system may recognize that the movement from location 35 to 37 is relatively small and the probe thus remains in the superior vena cava, and in particular a particular distance upward from location 35. Further, the system may use this data, in various respects referred to as expert data, to improve the information generated for the clinician. For example, the system may recognize that the structures 11, 13, 17, 18, and 19 are the same and use this knowledge when generating the image data at location 37. In various embodiments, the system may track and make use of position information. The catheter position may be monitored with position sensors. The catheter position may be tracked by monitoring the robotic motors. The system may include sensors for monitoring the position of the patient, and using this information in reference to the catheter.

In various embodiments, the system is designed to obtain image data at predetermined locations. The system uses the predetermined location information to identify anatomical landmarks. For example, at locations 35 and 37 in the superior vena cava, the system expects to have the right ventricle closest in the field of view and the left ventricle further in the field of view. Such information may be used to interpolate the image data.

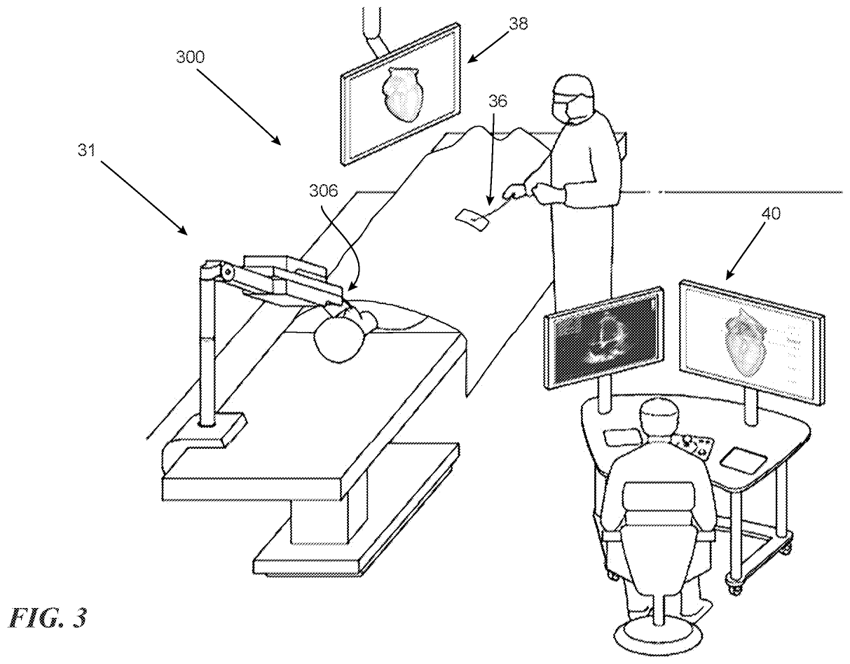

FIG. 3 is another illustration of a system 300 that can include a robotic positioning system 31 which can be coupled to an imaging system 306. The imaging system 306 can include, for example, an imaging probe coupled to a catheter, or a system similar in functionality to conventional TEE systems (e.g., for imaging the heart of a patient). The system 300 can further include a surgical device or system 36. For example, the imaging system 306 can be configured to generate images and/or real-time 3D models of the patient's heart, and the surgical device or system 36 can be any surgical device configured for treatment or therapy of the heart (e.g., such as those non-invasive surgical systems and devices used by interventionalist cardiologists). The system can include a display 38 configured to display real time imaging and/or a 3D model of the patient's heart (e.g., from imaging system 306), and can further include a console 40 configured to provide image processing, 3D modeling, and robotic control, among other features. The console 40 may include additional displays, as shown.

In various embodiments, the system uses data from multiple locations to construct a digital model. For example, the system may collect data from multiple locations in the superior vena cava to construct a 3D model of the surfaces of the myocardial wall and ventricles. In various embodiments, the model is static like a CT scan or MRI. In various embodiments, the model is dynamic and includes temporal data. In various embodiments, model is changing in real-time. In various embodiments, the model represents historical changes. For example, the model may show changes to the anatomical structure during a defined time period, e.g., during electrical stimulation of the heart.

The system may apply a variety of techniques to manipulate the data as would be understood by one of skill from the description herein. In various embodiments, the system applies statistical fit to identify anatomical landmarks based on data obtained at different locations. In one example, the system identifies discrepancies between an expected characteristic(s) and the observed or imaged characteristic(s). The system can use these discrepancies, in particular when accumulated over a plurality of data points, to improve fit of the data. In various embodiments, the system processes data using a technique similar to principal component analysis. Other suitable techniques include, but are not limited to, fuzzy logic, machine learning, and artificial intelligence (e.g., expert analysis).

In various embodiments, the system includes automation and/or robotics. In the example where images are taken at predetermined locations, robotics may be used to precisely control movement of the probe between those locations. In an exemplary embodiment, the clinician positions the probe at a starting reference point, for example location 35 in FIG. 2. The system then may be configured to automatically move the probe to the next location in the predetermined series. In various embodiments, the robotics may be used to automatically move the probe to desired locations. In various embodiments, the system is configured to guide the clinician between desired locations. For example, the system may display guidance information to the clinician during probe positioning. The guidance may be simplistic, such as indicating to move the catheter forward or back until a desired location is reached. The guidance may be more sophisticated, such as indicating landmarks for the clinician to use during positioning.

The system may include a control system configured to manipulate a position and/or orientation of the catheter and/or imaging element. This may be, for example, the console 40. In some embodiments, the control system may include or be operatively coupled to the automation and/or robotics described above. The control system and/or console may further include one or more processors and memory coupled to the one or more processors, the memory being configured to store computer-program instructions, that, when executed by the one or more processors control the movement of the catheter, probe, and/or robotics/automation to obtain a desired or optimized view. The control system may be configured to move the catheter and/or imaging element within the patient such that the imaging element obtains the desired view.

The computer-program instructions may include software including artificial intelligence and/or machine learning software. The software may include pre-trained classifiers. In some embodiments, the software may include instructions, that, when executed by the one or more processors, automatically classifies a present view of the imaging element and provides instructions to the control system to move the imaging element to a location that optimizes the desired view. The computer-program instructions may be further configured to apply a score to a present view of the imaging probe and/or catheter. In some examples, the computer-program instructions are further configured to provide instructions to the control system to move the imaging element until the score for the present view is above a target threshold. In other embodiments, the computer-program instructions are further configured to provide instructions to the control system to move the imaging element until the score for the present view is maximized.

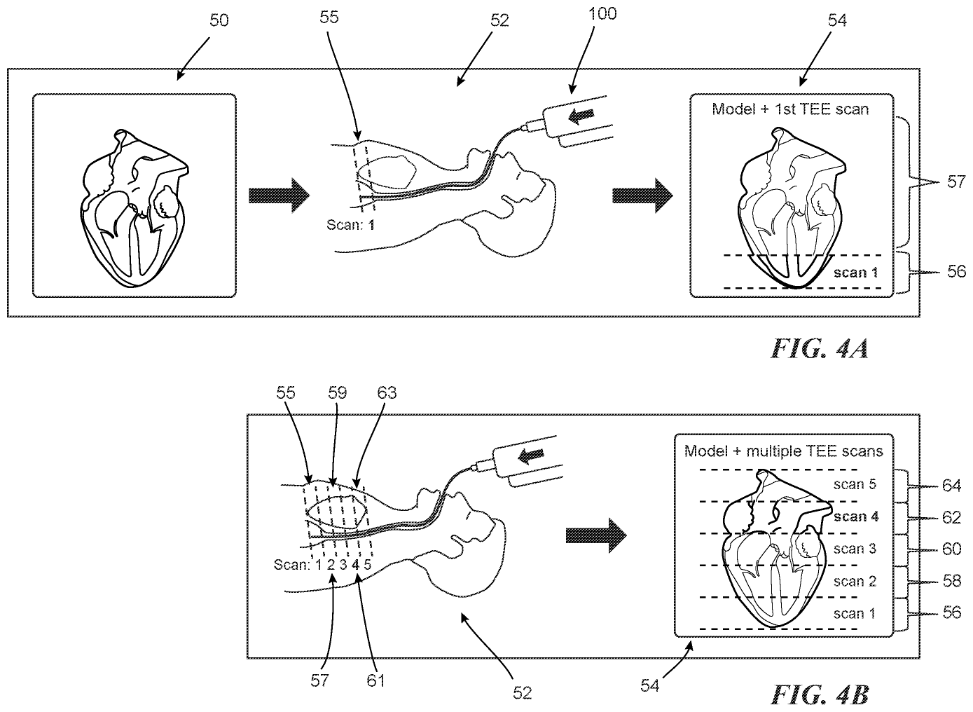

FIGS. 4A-4B illustrate one method for generating a 3D model of a patient's heart. At step 50, the software model can start with an initial 3D model of a heart based on a standard distribution of hearts. In some implementations, the distribution of hearts can be tailored to the patient's age, sex, disease state, and demographic. In some embodiments, the standard distribution can exclude outliers (e.g., hearts with tumors, univentricular, and other rare cardiac disorders). In other embodiments, the 3D model can start with actual imaging of the patient's heart instead of a standard distribution of hearts. For example, CT, MRI, or other high-definition imaging of the patient's heart can be used by the model as a starting point.

The 3D model of the heart can use a physics-model and can use a FEA mesh as a starting point for model construction. Optionally, a dynamic digital twin template can be stored in memory and modified based on imaging of the patient's heart (e.g., CT, MRI).

At step 52, the imaging system 100 can be positioned at location 55 to acquire a first scan or imaging slice of the target anatomy, such as the patient's heart. As shown in FIG. 4A, the imaging can be performed by a TEE system, or an imaging system with similar functionality (e.g., the probe 106 described above in FIG. 1). As shown, in some embodiments the first scan acquires an image of only a portion or slice of the target anatomy (e.g., the patient's heart). In some embodiments, the imaging system can include GPS or other position/real-time tracking so the system knows precisely where the image slices are collected relative to the target anatomy. The position of the probe or imaging system can be stored or recorded along with each image slice. In one example, a standard GPS coil can be used for positioning/tracking. In another embodiment, a GPS coil can be wrapped around the imaging system transducer(s) or probe(s) and either laminated or heat shrunk.

Next, at step 54, the system can update the initial 3D model of the heart using information from the first scan. As shown in FIG. 4A, only the portion 56 of the initial 3D model corresponding to the first scan is updated or modified. The remaining portion 57 of the initial 3D model remains unchanged. The portion 56 of the 3D model that is updated or modified is associated with the imaging or scan taken by the imaging system at location 55 from step 52.

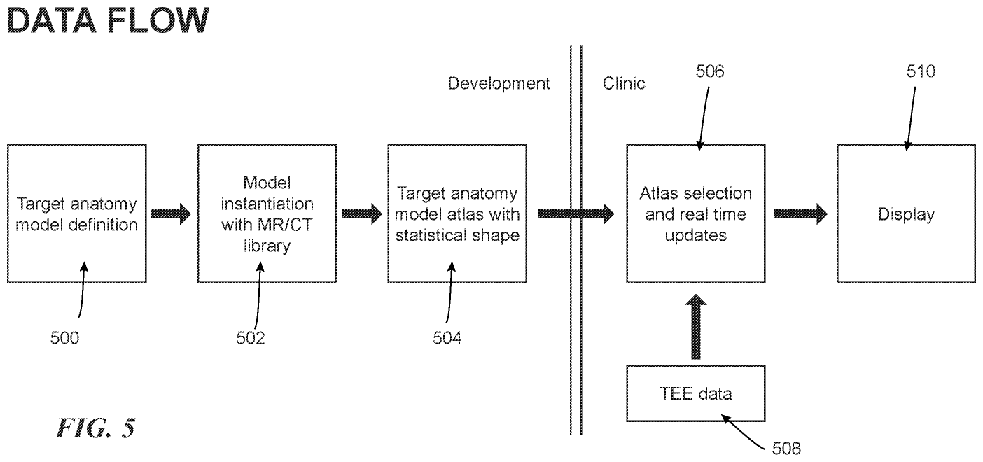

Referring to FIG. 4B, the method steps 52 and 54 of FIG. 5A can be repeated for multiple additional scans of the target anatomy (e.g., the heart), resulting in an updated 3D model of the heart after each subsequent scan. With enough scans, the entire 3D model of the target anatomy (e.g., heart) can be updated with the real-time imaging information from the imaging system. In the embodiment shown, the target anatomy is divided into five different portions or scans 56, 58, 60, 62, and 64, corresponding to images or scans taken at locations 55, 57, 59, 61, and 63, respectively. It should be understood that the number of scans or portions of the target anatomy can be customized or changed within the system. For example, dividing the target anatomy into more scans may result in a higher resolution or more accurate 3D model at the expense of a longer procedure since more scans or slices need to be taken and processed. The plans of the scans may be changed such that the scans are not parallel.

FIG. 5 is a data flow chart corresponding to the method steps described above in FIGS. 4A-4B. The steps of the method of FIG. 5 can be implemented in one or more computing systems. In some embodiments, the computing system can be incorporated into an imaging system such as the imaging system 100 described above. In other embodiments, the computing system can be separate from the imaging system 100 but in communication with the imaging system.

As shown in FIG. 5, the 3D model development can start at step 500 with a target anatomy (e.g., heart) model definition. At this step, the type of model can be determined or selected, including for example solid modeling, wireframe modeling, or surface modeling.

At step 502, the method can include model instantiation with a library of MR/CT or other high resolution medical images of patient's hearts.

Next, at step 504, a heart model atlas can be generated with a statistical shape based on the imaging library from step 502.

In use (in the clinic), at step 506, an atlas can be selected for the patient and updated in real time with the TEE (or other real-time imaging) data at step 508 (e.g., using the techniques described above and particularly in FIGS. 4A-4B) and displayed to the user at step 510. Reconstruction of the 3D model in real-time can be accelerated be reducing the number of possible shapes in the model. The model can be a cyclic model. If the target anatomy is a human heart, repeatability of the heart cycles can be used by the system to predict the anatomy position, so the entire heart can be continuously displayed.

The 3D model of the target anatomy, and the procedure, can then be presented on a display at step 510 to the physician or surgeon.

The model of the target anatomy (e.g., the heart) can include texture, tissue stiffness, movement, and/or other physical or physics-based parameters that can be used by the system or by a physician during a procedure to assist or improve the procedure. For example, the 3D model can be used to generate a haptic feedback model for the physician during a procedure. In use, the system and 3D model/haptic model can provide additional or enhanced haptic/audio feedback to the physician. For example, the system can provide feedback to the physician when the tool or therapeutic is positioned properly, or when it passes a notable or key portion of the anatomy. Additionally, feedback can be provided when providing therapy or when the therapy/procedure is completed.

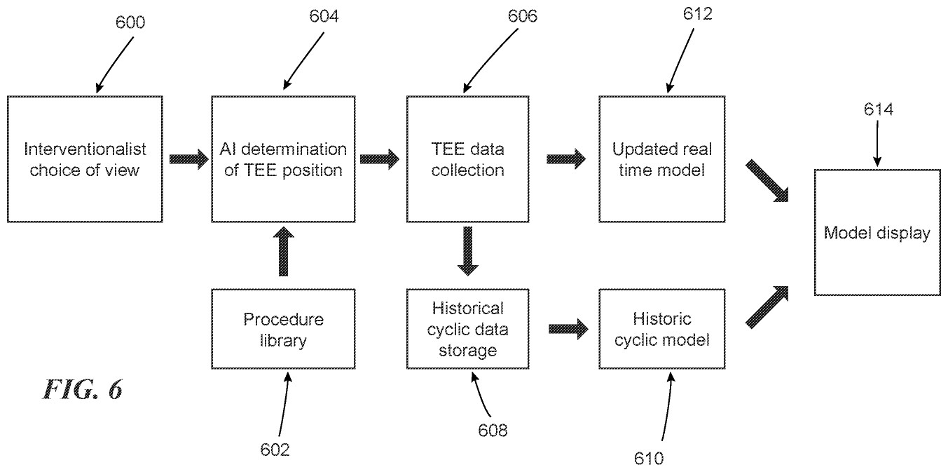

FIG. 6 is a flowchart that shows a top level algorithm/process flow for using the systems and methods described herein. First, at step 600, the physician (such as an interventionalist) can select or choose the type of view of the anatomy/procedure. This can include, for example, a 3D model of the target anatomy, 2D model of the target anatomy, color/black and white, cross-sectional views, etc. At step 602, the physician can further select a procedure type from a procedure library and whether or not to include the tool/device that is being used in the model. This can be selected/chosen at the start of imaging. In another embodiment, the specific size of the device can be chosen by the user to be displayed/used by the system. In some embodiments, selecting the procedure type can automatically determine the optimal view type for that procedure.

Next, at step 604, the system (e.g., the algorithms/artificial intelligence software) can determine the position and/or orientation of the TEE system (or other real-time imaging) relative to the target anatomy. This can be based, for example, on the procedure library from step 602 that contains data from previous patients and procedures.

At step 606, the TEE system (or other imaging system) can acquire imaging data of the target anatomy. At steps 608 and 610, as the TEE system acquires image slices of the target anatomy (e.g., the patient's heart), the data can be store in the historical imaging database and a historical cyclic model can be updated. Additionally, at step 612, the 3D model of the target anatomy can be updated in real-time with the TEE images (e.g., with the process shown in FIGS. 4A-4B). Finally, the 3D model can be displayed to the physician at step 614.

Methods of using the systems described herein are within the scope of the inventions. The system data may be used for pre-operative planning or interoperative guidance. The system may be configured as a diagnostic tool or supplemental to a therapeutic.

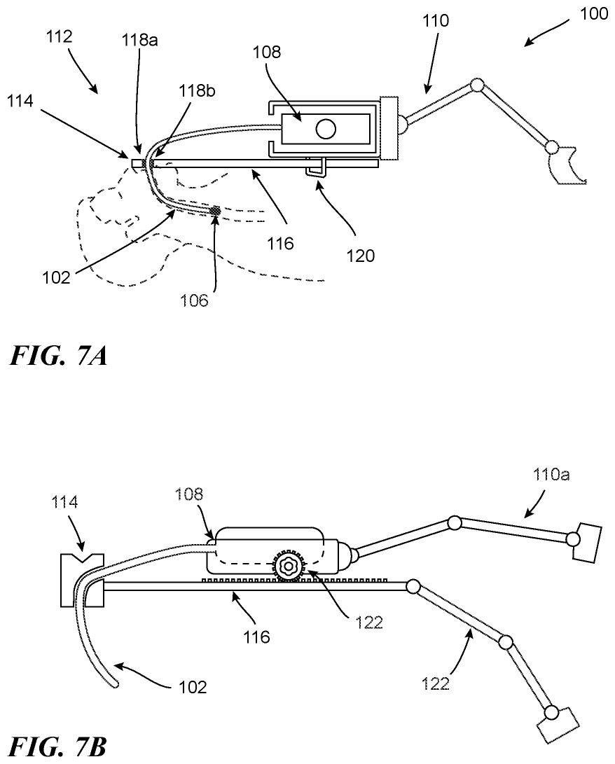

In one embodiment shown in FIG. 7A, a system 100 is shown for imaging a part of an anatomical structure (e.g., a heart). As described above, the system 100 can include at least a catheter 102 configured for insertion into a patient's body connected to a console positioned outside the body (not shown). In various embodiments, the catheter and console in accordance with the invention may be similar to conventional TEE, transthoracic, and other medical imaging systems. The system includes a probe 106 positioned on the end of a catheter for positioning inside the body.

The probe 106 may be one of a variety of imaging modalities. Examples include an ultrasound transducer. Such imaging probes may be known by one of skill in the art from the description herein including, but not limited to, conventional probes used for transthoracic and TEE. In some embodiments, the system can include a plurality of imaging modalities and can be configured to multiplex through the different imaging modalities to acquire the necessary images (e.g., TEE+transthoracic probes).

The probe can be electrically connected to the console. Processing of the data may be accomplished via software in the console. Various types of probes may be used as would be understood by one of skilled in the art.

In various embodiments, the system including the catheter and probe may be automated, such as by robotic control. For example, the probe and/or catheter may be coupled or attached to a robotic surgical system or robotic positioning system, that may include one or more robotic arms. Other embodiments are contemplated in which a robotic system is not required for manipulation of the catheter and/or probe. In FIG. 1A, a handle 108 of the catheter/probe is shown coupled to a robotic arm 110 of a robotic positioning system. The robotic arm can be configured to advance the catheter and/or probe distally/proximally into and out of the patient, and can be further configured to translate or rotate the catheter and/or probe. Additionally, the robotic arm can be configured to actuate controls of the probe, such as to capture images with the probe. In various embodiments, the system is configured to store and interpret data taken by the probe in multiple locations. In various embodiments, the system uses the interpreted data to generate information for a clinician. For example, the system may construct a 3D anatomical model based on image data taken from multiple locations. In another example, the system generates image data based on composite data from multiple locations.

The system 100 can further include a disposable 112 configured to operate as an interface between the probe and the robotic arm of the robotic positioning system. In some embodiments, the disposable 112 can include a number of components, including an interface and sensing component (ISC) 114 configured to be inserted into a mouth of the patient and a connecting member 116 configured to couple the ISC to the robotic arm. The ISC 114 can include a hole or lumen configured to receive the catheter and the probe. The probe and catheter are introduced in the patient's body by a physician or nurse or assistant. The ISC is configured, for example in the TEE configuration, as a mouth piece, bite lock, or bite guard, connected to the patient's mouth. In some configurations the ISC can be configured to attach to the patient's leg at a location proximate to the catheter insertion access site. The ISC provides a physical link between the patient and the robot. The ISC can be instrumented with one or more sensors 118a (e.g., displacement sensors) to track the travel and/or rotation of the catheter as it moves through the ISC, therefore determining the location of the transducer inside the patient's body. The sensors can further comprise force and torque sensors to track the forces being exerted on the transducer by the patient's anatomy. The intent is to prevent high forces being exerted on the patient by the transducer potentially causing harm to the patient as, for example, an esophagus tear.

Additionally, the disposable can further include one or more sensors 118b positioned on the connecting member 116. The sensors can comprise, for example, sensors configured to measure axial travel of the probe, rotation/orientation of the probe, and/or force sensors (e.g., measuring the force applied by the probe to the mouthpiece or the connecting member).

As described above, the connecting member 116 can be configured to rigidly couple the mouthpiece to the robotic arm. In one embodiment, the connecting member 116 can comprise a shaft, rail, or track configured to interface with an engagement member 120 disposed on either the robotic arm or the handle of the probe. In some examples, the connecting member can be configured to slide along or within the engagement member. The interaction between the connecting member and the engagement member can prevent excessive forces from being translated from the robotic arm to the patient and/or to the mouthpiece.

FIG. 7B illustrates an alternative embodiment of a connecting member 116 and engagement member 122 that can comprise a rack and pinion arrangement. As with the embodiment of FIG. 7A, the catheter 102 can be coupled to a handle 108, which can be coupled or attached to a robotic arm 110. The connecting member can be attached or connected to the ISC 114 (e.g., the mouth piece) and also to a mounting arm 122 which can optionally be a second robotic arm. The engagement member 122 can couple the handle 108 to the connecting member 116. As is known in the art, the pinion (engagement member 122) can convert rotational motion into linear motion along the rack (connecting member 116) which can include teeth configured to interface with teeth of the pinion. When the robotic arm 110 is translated axially, the handle 108 and catheter 102 move axially along the connecting member 116. In some embodiments, axial movement of the catheter can be tracked with one or more sensors in the ISC as described above. Alternatively, sensors on the connecting member or rotation of the engagement member 122 can track axial movement of the catheter. In some embodiments the handle and catheter can be configured to rotate with respect to the connecting member 116. For example, the engagement member can sit in a pivot or groove in the handle to facilitate rotation of the handle while still allowing for axial movement along the connecting member 116.

According to one embodiment, a pre-operative heart model is created by the system using sample heart images, from ultrasound, MRI or CT. This model can then be virtually represented in the system with geometric primitives. This virtual representation can be in the form of closed b-spline explicit surfaces to describe the different chambers and valve leaflets of the patient's heart. Alternatively, the geometric shape can be described by a tessellated mesh.

FIG. 8 illustrates a flowchart describing a method of acquiring images and generating a 3D model of a patient's heart. The method can be implemented using any of the systems described herein. In one example, the physician or user can activate a scanning and registration procedure on the robot. The robot and/or user moves the transducer in such a way as to scan the target anatomy (e.g., the heart). The transducer is configured to acquire sufficient data to fit the virtual model creating a patient specific model. The model is then rendered by the system and an image is displayed on the display screen. This process is described above with respect to FIGS. 4A-4B, for example.

Following the initial scanning step described above, the system can then move to an iterative state. The physician selects a pre-determined standard TEE named view from a menu. At step 802, the probe can take an image at the current position of the probe. At step 804, the current image obtained by the current position of the probe is analyzed with a trained neural network image classifier. If the image meets the goodness of fit criterium the image is displayed, and the system waits for another input from the physician. If the image does not meet the goodness of fit criterium, at step 806, a navigation module is activated. In this module, a new probe position is determined, and the appropriate kinematic solution is sent to the robot. Once the probe reaches the new position, a new image is taken with the probe, the image is analyzed again, and the loop is restarted. When all the images for each standard TEE named view meet the goodness of fit criterium, the 3D model can be reconstructed (step 808), for example by using point clouds, polygonal models, or parametric surfaces. The 3D model can then be displayed to the user at step 810. The displayed model can be a photo realistic rendering, e.g., a 3D model plus textures applied to the model.

In some embodiments, a statistical fit is provided where the system updates the model based on a comparison between the actual image and the expected image.

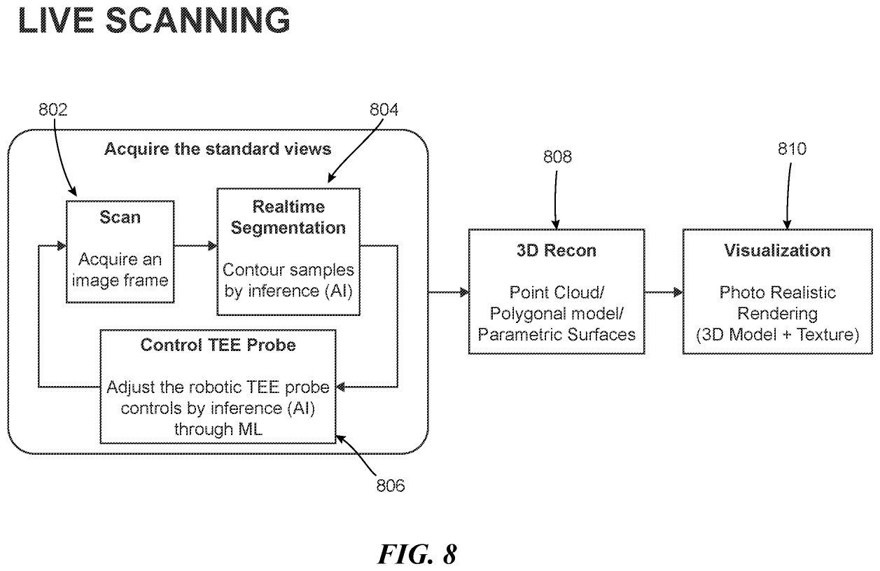

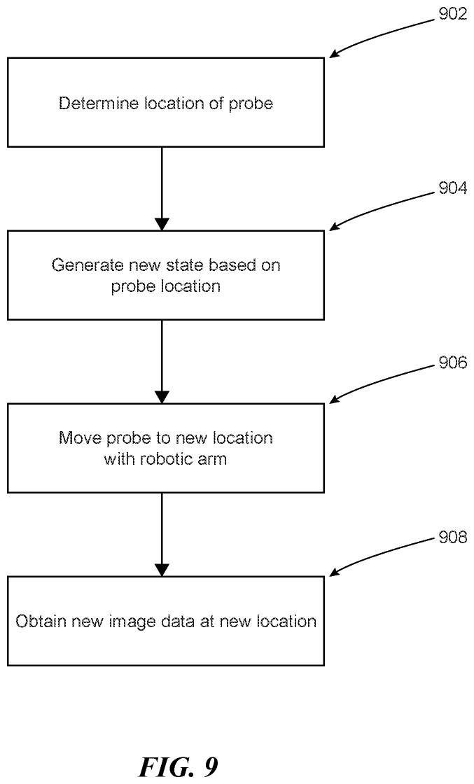

In another embodiment, referring to FIG. 9, the method can include updating the 3D model based on a device state. For example, the system can, at step 902, determine a location of the probe, and at step 904, generate a new state based on the probe location. This determination can be made using the sensors described above. Next, at step 906, the system can move the probe with the robotic arm to a new probe location, and at step 908 obtain new image data. This process can then be repeated to update the 3D model.

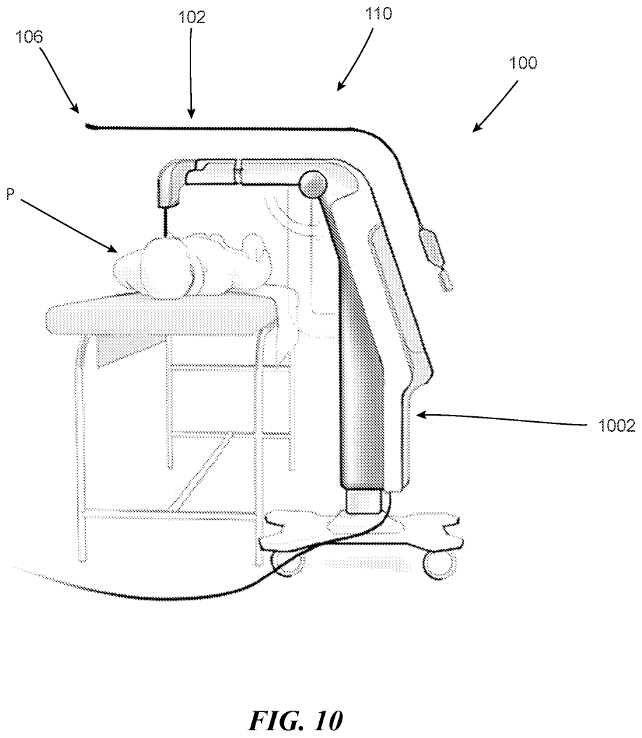

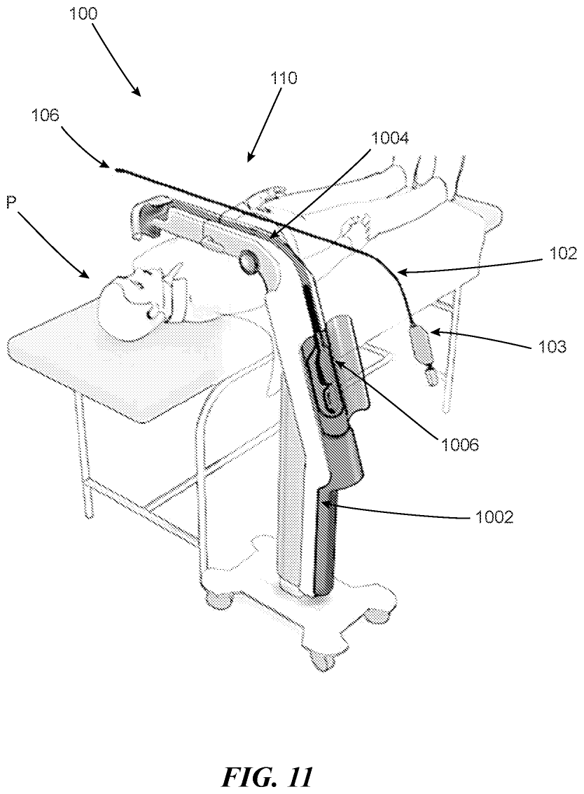

FIGS. 10-13D show an exemplary procedure using the system in accordance with aspects of the disclosure. Referring to FIGS. 10-11, a patient P is positioned on a table in in operating room. A system 100 including a robot 110 is positioned next to the patient. The robot includes wheels so it can be moved into position in between different operating rooms or labs. In various embodiments the robot is mobile and can be rolled between different locations or taken apart for easier movement.

A system 100 includes a robot 110 having a base which includes wheels in the exemplary embodiment. The exemplary robot is formed as a tower in the illustrated form factor. A main support 1002 rises from the base and an arm 1004 extends over the patient and the table. The arm can be pivotable or otherwise moveable with respect to the main support. The arm can include an imaging configuration, for example in which it is extended from the support tower. In some embodiments the arm can be moved (e.g., swung) prior to, during, and/or following an imaging procedure. Movement of the arm (while maintaining the position of the robot base) enables unobstructed access to the patient, for example while imaging with the present system is not being performed. The arm can include a mobile configuration, in which it is placed adjacent or proximate the main support to reduce its overall spatial footprint and/or improve stability for movement.

An imaging catheter having a probe 106 is connected to the robotic arm and the system. The robotic arm includes a cartridge interface for receiving a handle of the imaging probe. A distal end of the imaging probe including the transducer extends out of the end of the robotic arm. The robotic arm (or other portion of the system) may include elements designed to control buckling of the catheter (e.g., anti-buckling) during use thereof. In the exemplary embodiment the robot can receive and manipulate any off the shelf imaging probe. In alternative embodiments the imaging catheter is integrated with the robot itself.

The probe 106 can use various imaging modalities as would be understood by one of skill in the art including, but not limited to, ultrasound, CT, and MRI. In an exemplary embodiment, a transesophageal echo (TEE) probe is connected to the robot.

The exemplary catheter has a handle 108 configured for placement in a robotic receiving cartridge 1006 and the opposite (distal) end with the probe configured to be positioned within the esophagus of the patient. A cartridge which receives the handle of the probe can have a clamshell design to snap shut around the handle thereby securing it in place. The robot is configured to manipulate the handle in the same fashion as an expert clinician. The robotic system includes various motors for manipulating the handle controls. For example, the robotic arm can include servo motors driving wheels, levers, and the like. In this manner, the robot is configured for axial translation and rotation of the catheter and/or probe.

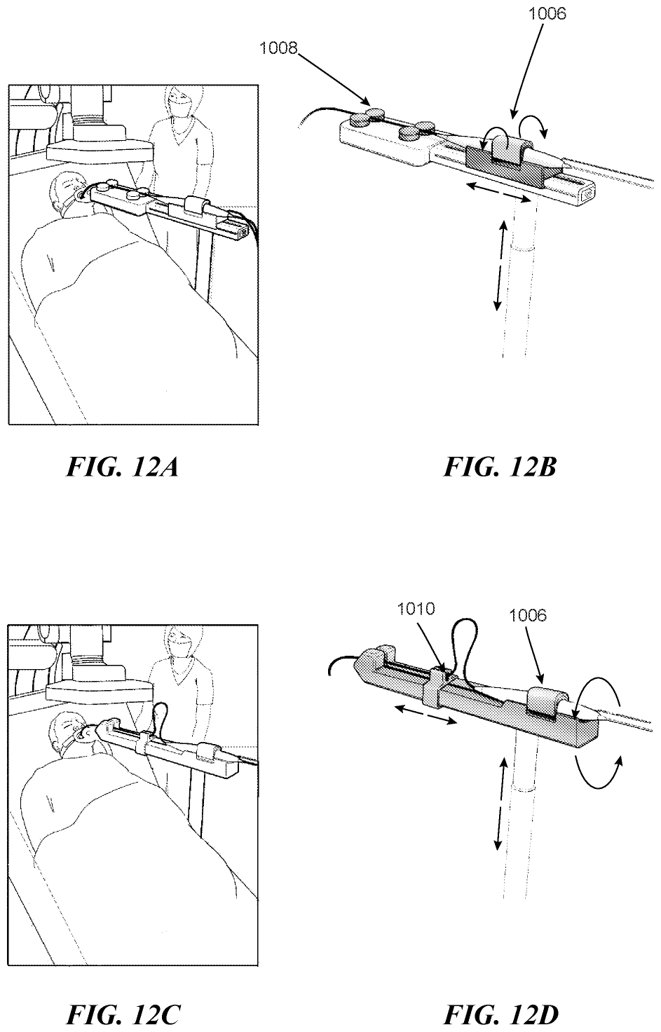

With particular reference FIGS. 12A-12B, an embodiment of the system includes a driven pulley system. The pulley system includes drives or wheels 1008 configured to interact with the catheter which turn thereby translating or manipulating the catheter. The system can include a cantilevered design so the probe can be positioned close to the mouth of the patient. An exemplary horizontal cartridge 1006 engages the handle controls. The robot includes a stage mounting the cartridge. The stage is configured so it can translate in the X axis, Y axis, Z axis and rotate, thereby moving the catheter and probe.

With particular reference to FIGS. 12C-12D, an alternative linear drive system is shown. The system includes a carriage 1010 which clamps part of the catheter and translates in and out. On a proximal end the entire body of the cradle 1006 can rotate the handle and therefore the catheter/probe.

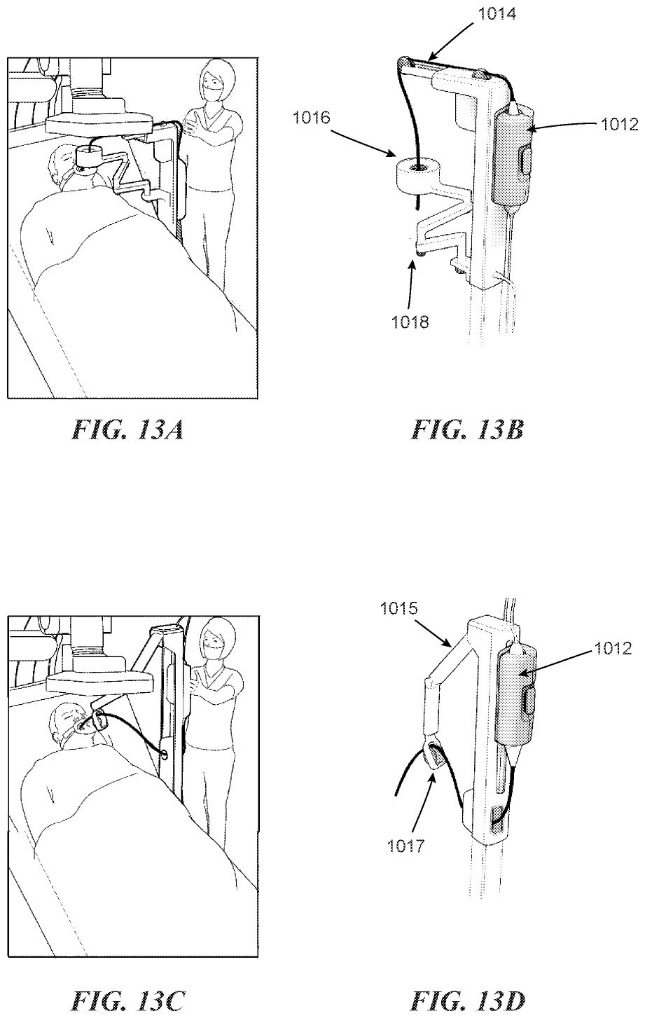

With particular reference to FIGS. 13A-13B, an alternative guide arm system is shown. The system includes a vertically mounted cradle (carriage) 1012 which clamps the handle of the catheter for control of rotation thereof. The cradle further engages with knobs (e.g., controlling distal flexion of the catheter) and buttons of the handle for control thereof. An extendable track 1014 supports a middle portion of the catheter and controls catheter depth, while a generally annular interface 1016 is positioned at the end of a multi-axis adjustable arm 1018 to move the catheter in an X-Y plane.

With particular reference to FIGS. 13C-13D, an alternative vertical rail system is shown. The system includes a vertically mounted cradle (carriage) 1012 which clamps the handle of the catheter for control of rotation and translation (e.g., depth) thereof. The cradle further engages with knobs and buttons of the handle for control there. A robotic arm 1015 supports a middle portion of the catheter and controls the position of the shaft with respect to the patient's mouth via a pulley wheel 1017.

The cradle and robotic controls are driven by a controller in the robot. Various other mechanisms and sensors may be employed. For example the system may include position sensors for discerning the position and movement of the robotic controls. The system may include force feedback sensors to reduce the risk of the robot pushing against tissue structures and causing perforation. The position sensors may also be used for guidance as would be understood by one of skill in the art.

The console includes a display port showing and representing the image data from the catheter. The console may include other features in the presentation layer. For example the console may show a 3D model of the anatomy, in this case the heart.

In various embodiments the system makes use of preoperative information. In various embodiments a CT scan or other imaging data is provided as part of the planning process. For example a CT scan is part of the normal protocol for many types of procedures. The system can make use of this data for operation. In various embodiments the system is configured to generate and update a 3D model of the heart to guide the interventional procedure. The system may start with a base 3D model based on existing data sources. The model may be updated based on the personalized CT scan of the patient.

The robot drives the catheter using the handle similar to a clinician. Described below are various control schemes for driving the catheter.

In one embodiment the catheter is positioned in the patient such that the probe is generally positioned at a predetermined point in the esophagus. In various embodiments the probe is positioned in the center of the esophagus. In various embodiments the probe is positioned in the upper end of the esophagus. In various embodiments the probe is positioned near the lower sphincter of the esophagus. In various embodiments, the probe is positioned where it has a central view of the heart of the patient.

Once the probe is in position the user can start the robotic action.

The robot can operate in different ways as would be understood by one of skill from the description herein.

In various cases the robot starts by performing an initialization. The robot moves the probe up and down in the esophagus while recording image data. Throughout this process or after an initial or initial set of passes, the robot updates a 3D model of the heart. This may be in addition to or instead of incorporating the preoperative image data as described above. This process also allows the robot to build a map which may be used as a reference for further navigation of the robot.

In various cases once the probe is in position the robot may be put into immediate use without the above process.

The robot may be used in different ways in operation. In various embodiments the clinician may interact with the robot in a similar way as the interactions with an expert echocardiographer. For example the console may include controls so the clinician can request particular (standard) views. In another example the system is pre-programmed to guide certain procedures. For example a clinician may push a button for a left atrial appendage (LAA) closure procedure or transcatheter aortic valve implantation (TAVI) procedure and the robot makes use of expert knowledge to anticipate these several views that will be needed to guide the procedure. In this case the system may automatically move to one or more predetermined locations to collect these image data before the procedure begins, to provide faster response and better imaging intraoperatively.

The robot can be driven in a variety of ways. The system may include a microprocessor, ASIC, FPGA, and/or other hardware. The system may be pre-programmed with a control scheme, algorithm, or other manner. The system may be driven autonomously or with learning. For example the system may be configured for deep learning during the procedure. Deep learning may be used to refine any algorithm used in image gathering and/or display, including robotic control of the imaging probe.

In an exemplary embodiment the robot is driven by a controller which incorporates an algorithm. The robot drives the probe via movement of the cartridge in incremented steps. For example it may translate in increments of half a centimeter (0.5 cm) over a predefined distance (e.g., 20-40 centimeters). The probe may be rotated as it is translated. For example the probe may be rotated 45-90 degrees, incrementally, to scan the heart as it's translated. The robot may be rotated in predetermined (e.g., 1-5, inclusive) degree increments. The robot may be driven in various other patterns and record image data as it goes.

In various embodiments the system makes use of computer vision to identify structures and recognize image views. In an exemplary embodiment the system is used to guide a structural heart procedure. As the probe is driven along the esophagus the system uses image recognition to identify portions of the heart. For example the system recognizes and identifies the four chambers of the heart, the valves, the coronary arteries, and the lungs. In various embodiments as the system is moving and collecting the image data it recognizes the anatomical landmarks. The system may be programmed to stop at these locations and record the catheter configuration (e.g., position of cradle, track, handles, knobs, and ultrasound settings) into the system registry. The system may further be configured to optimize the image by moving locally in this region to achieve the best image. The best image may be identified using machine learning techniques.

In various embodiments, the system is driven somewhat autonomously. The system may be self-driving. The system may employ techniques such as a “hunt and peck” method or recursive method. Any form of the system uses complex algorithms for learning to navigate. For example the system may make use of fuzzy logic whereby it manipulates the probe through the anatomy all the while recognizing structures as it goes. For example it may recognize that if it started at the top of the esophagus and moved a certain distance it would be near the middle of the esophagus. It would further recognize that if it moved even further it should eventually encounter the sphincter of the esophagus. The system may optionally incorporate other information such as force feedback. For example if the probe starts at the top of the esophagus and encounters resistance it may recognize that it's encountered a wall thereof. In a recursive approach, the system may be program to move until a predefined event or trigger occurs. For example the system may move down the esophagus until it achieves the optimal image of the mitral valve.

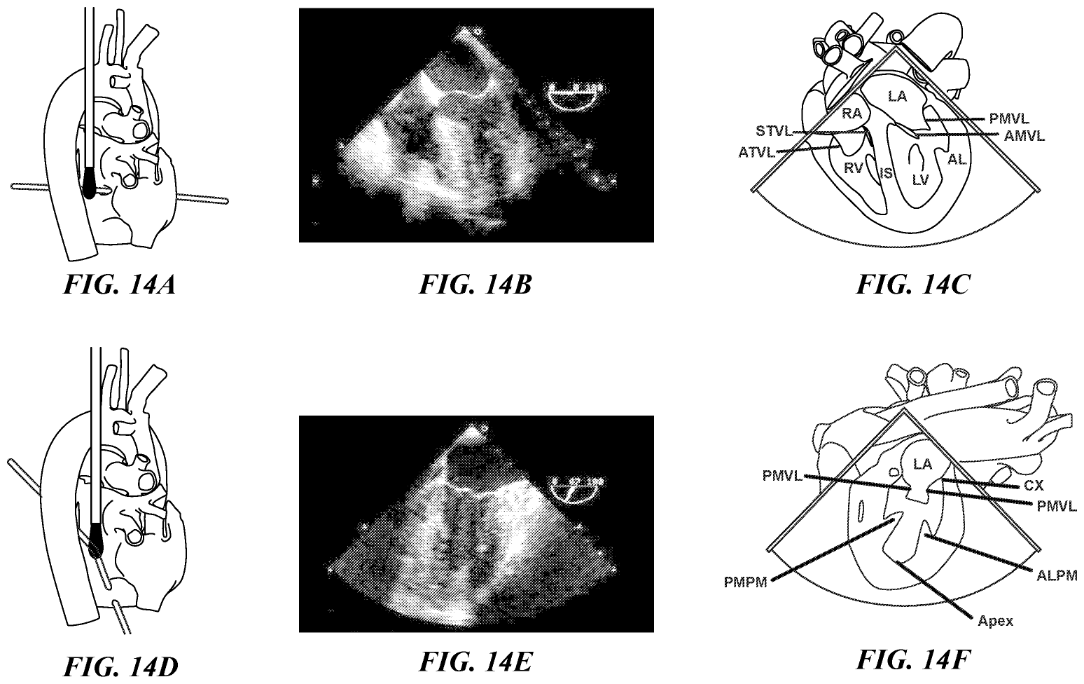

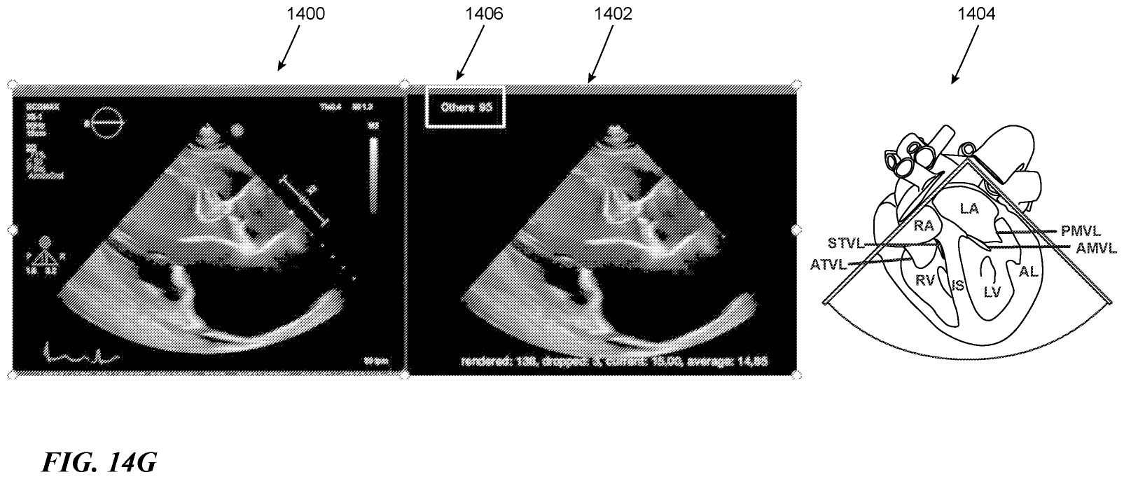

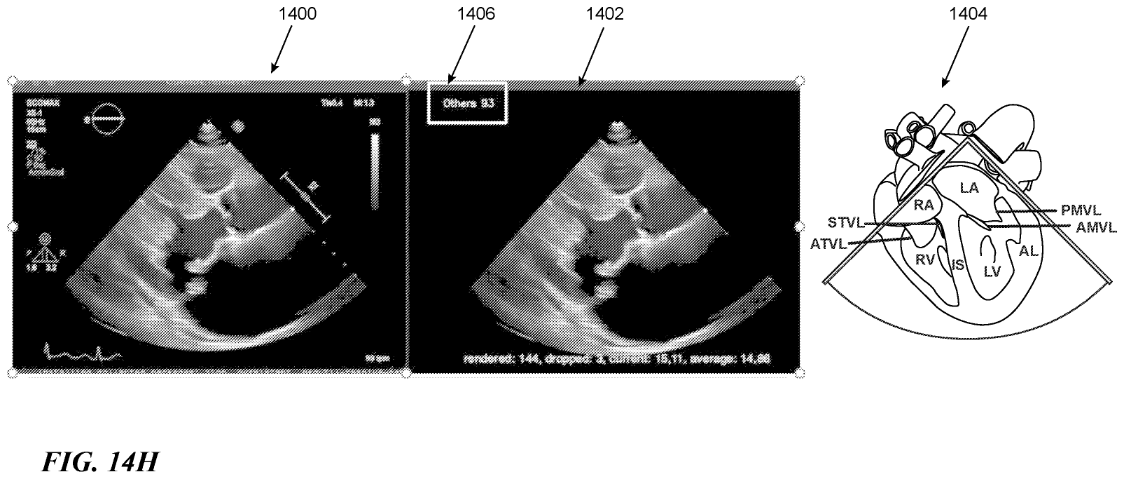

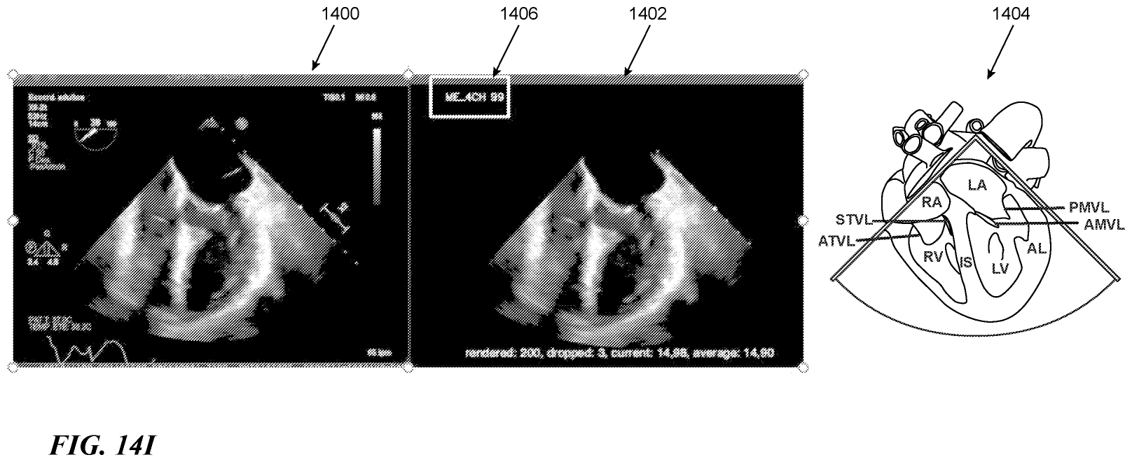

As described above, a 3D model of a target anatomy (e.g., a heart) can be generated and updated based on image slices taken with an imaging probe, such as the system 100 which can be for example a TEE probe system. Various probe positions and slice orientations can make up a library of views within the 3D model of the target anatomy. For example, FIG. 14A illustrates a position of a probe 106 with respect to the target anatomy taking an image slice 1401 corresponding to a standard “Mid-Esophageal Four Chamber” TEE view. FIG. 14B shows the actual ultrasound image slice 1401, and FIG. 14C illustrates a cross-sectional slice of the 3D model of the target anatomy corresponding to the Mid-Esophageal Four Chamber Tee view. FIGS. 14D-14F illustrate a position of a probe 106 with respect to the target anatomy taking an image slice 1403 corresponding to a standard “Mid-Esophageal Mitral Commissural” TEE view. As with above, FIG. 14E shows the actual ultrasound image slice 1403, and FIG. 14F illustrates a cross-sectional slice of the 3D model of the target anatomy corresponding to the Mid-Esophageal Mitral Commissural Tee view. While these views are shown merely for illustrative purposes, it should be understood that a 3D model of a heart using standard TEE views may include some or all of the standard TEE views, additionally including a Mid-Esophageal Two Chamber view, a Mid-Esophageal LAX view, a Mid-Esophageal Aortic Valve SAX view, a Mid-Esophageal Aortic Valve LAX view, a Mid-Esophageal Right Ventricle Inflow-Outflow view, a Mid-Esophageal Bicaval view, a Mid-Esophageal Descending Aortic SAX view, a Mid-Esophageal Descending Aortic LAX view, a Transgastric Mid SAX view, a Transgastric Two Chamber view, a Transgastric Basal SAX view, a Transgastric LAX view, a deep transgastric LAX view, a Transgastric Right Ventricular Inflow view, an Upper Esophageal Aortic Arch LAX view, an Upper Esophageal Aortic Arch SAX view, a Mid-Esophageal Ascending Aorta LAX view, and/or a Mid-Esophageal Ascending Aorta SAX view.

FIGS. 14G-14I illustrate a method of moving a catheter and probe according to some embodiments of the disclosure. As described herein, an initial scan of a target anatomy with the system described herein can be used to update or modify a 3D model of the target anatomy.

Referring to FIG. 14G, views 1400 and 1402 illustrate ultrasound image slices taken of a target tissue anatomy (e.g., a heart) from a probe at a given or present position (e.g., the probe of the systems described herein). In this embodiment, a user of the system has selected a Mid-Esophageal Four Chamber view as the desired or selected view of the system. View 1404 illustrates a standard TEE Mid-Esophageal Four Chamber view (ME_4CH) from a 3D model generated by the system from a library of ultrasound image slices of the target tissue anatomy (e.g., the heart). This is the desired or selected view from the user. View 1400 illustrates a current or present slice view of the target tissue anatomy based on the current or present location of the imaging probe. The current or present view of the imaging probe can be evaluated with software, such as machine learning or artificial intelligence software to apply a score to the current or present view. In some examples, the score provides a confidence level 1406 determined by artificial intelligence/machine learning of the system as to if the ultrasound slice or view 1400 is of the desired or selected view. In this instance, view 1402 indicates with 95% confidence that the view or slice shown in view 1400 is not in the 3D model library and is not the desired or selected view.

Referring to FIG. 14H, the probe has been moved, and views 1400, 1402, and 1404 are again shown. In this instance, scoring from the machine learning software, as illustrated in view 1402, indicates now with 93% confidence that the view or slice shown in view 1400 is not in the 3D model library and is not the desired or selected view. Since the confidence level has gone down from 95% to 93% with the probe movement, this suggests that the prior probe location (from FIG. 14A) was in a better position for the “Other” view which is not yet in the 3D model library. The probe can be moved again, and if the confidence range fluctuates closely (say between 93-95%), it can be a suggestion that the probe is in the correct location for the “Others” view. The system can bookmark this probe location for the “Others” view.

Referring to FIG. 14I, the probe can be moved again, this time showing views 1400, 1402, and 1404. Here, in view 1402, the software scoring model provides a confidence level 1406 of 99% that the ME_4CH view has been recognized, and that the current position of the probe and orientation of the probe (e.g., the orientation of the slice from the probe) aligns with the selected or desired ME_4CH view. In some embodiments, the software can determine that the current position and/or orientation of the probe is in the proper or optimized location when the score is above a threshold (e.g., above 95% confidence, above 96% confidence, above 97% confidence, above 98% confidence, or above 99% confidence). In other embodiments, the proper or optimized position can be determined by taking the maximum confidence level score over the course of probe movement. This view corresponds with the view 1404 shown from the 3D model. The image slices taken at this position can be used to further update or modify the 3D model relating to this standard TEE view or slice. In some embodiments, or in addition, instead of displaying a 2D image 1404, a displayed image can be a 3D reconstruction of the heart comprising the information associated with the images 1402.

Although described in terms of a cardiac model and positioning in the superior vena cava, one will appreciate that the system and method may be applied in a number of applications. For example, the system may be applied to other structures and systems in the body. The system may be used for imaging and/or analysis of the gastrointestinal system or renal system. In various embodiments, the system may be used to monitor functions. Examples include, but are not limited to, monitoring blood flow or interstitial fluid buildup. The system may be used outside the body, for example for an obstetric ultrasound.

In various embodiments, data from other sources may be incorporated into the system to improve performance and capabilities. For example, the system may incorporate personalized patient data. In one example, the system incorporates gender data to identify anatomical structures. In another example, the system incorporates CT data from the patient.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

What is claimed:

1. A method of automatically building and/or updating a cardiovascular model, comprising:
obtaining first image data from a first location in a patient, the first image data including information related to at least one anatomical structure;
obtaining second image data from a second location in a patient, the second image data including information related to the at least one anatomical structure; and
generating a representation of the at least one anatomical structure based on the first and second image data.

2. The method according to claim 1, wherein the generating includes building a representation of a 3D anatomical model.

3. The method according to claim 2, further comprising:
obtaining third image data relating to the at least one anatomical structure;
determining a correspondence between the third image data and the 3D anatomical model;
identifying a discrepancy between the at least one anatomical structure in the third image data and the associated at least one structure in the 3D anatomical model; and
updating the 3D anatomical model based on the discrepancy.

4. An imaging system for use in modelling an anatomical structure of a patient, comprising:
a catheter sized and shaped for percutaneous insertion into the patient;
an imaging probe having a field of view, coupled to the catheter near a distal end thereof;
a drive mechanism coupled to the catheter and/or the imaging probe, configured to translate and/or rotate the imaging probe; and
a processor operatively coupled to the imaging probe and the drive mechanism, the processor configured to transmit and receive signals with the drive mechanism and the imaging probe, to—
control the drive mechanism to place the imaging probe at a first and a second position within the patient, and
control the imaging probe to generate first image data and second image data related to respective first and second fields of view therefrom,
wherein the processor is configured to generate and/or update a model of the anatomical structure considering the first and second image data.

5. A method of automatically building and/or updating a cardiovascular model, comprising:
generating an initial 3D model of at least one anatomical structure based on a standard distribution of corresponding anatomical structures;
obtaining first image data from a first location in a patient, the first image data including information related to a portion of the at least one anatomical structure;
obtaining second image data from a second location in a patient, the second image data including information related to a different portion of the at least one anatomical structure; and
generating a modified 3D model of the at least one anatomical structure based on the first and second image data and the initial 3D model.

6. The method of claim 5, wherein the at least one anatomical model comprises a heart.

7. The method of claim 5, wherein obtaining first and second image data comprises obtaining first TEE image data and second TEE image data.

8. The method of claim 5, further comprising updating the standard distribution of corresponding anatomical structures with the first and second image data.

9. The method of claim 5, further comprising including a model of a selected therapeutic procedure or tool in the modified 3D model.

10. The method of claim 5, wherein generating the initial 3D model further comprises generating an initial 3D physics model of the at least one anatomical structure.

11. A non-transitory computing device readable medium having instructions stored thereon that are executable by a processor to cause a computing device to perform the method of:
obtaining first image data from a first location in a patient, the first image data including information related to at least one anatomical structure;
obtaining second image data from a second location in a patient, the second image data including information related to the at least one anatomical structure; and
generating a representation of the at least one anatomical structure based on the first and second image data.

12. An imaging system for use in modelling an anatomical structure of a patient, comprising:
a robotically-controlled drive mechanism configured to be coupled to and drive an imaging probe; and
a processor operatively coupled to the imaging probe and the drive mechanism, the processor configured to transmit and receive signals with the drive mechanism and the imaging probe, to—
control the drive mechanism to place the imaging probe at a first and a second position within the patient, and
collect image data from imaging probe at the first and second positions,
wherein the processor is configured to generate and/or update a model of the anatomical structure considering the first and second image data.

13. A method of automatically building and/or updating a cardiovascular model, comprising:
obtaining in a non-transitory computing device readable medium first image data from a first location in a patient, the first image data including information related to at least one anatomical structure;
obtaining in the non-transitory computing device readable medium second image data from a second location in a patient, the second image data including information related to the at least one anatomical structure; and
executing instructions stored in the non-transitory computing device readable medium with a processor to cause the computing device to generate a representation of the at least one anatomical structure based on the first and second image data.

14. A method of building and/or updating a cardiovascular model, comprising:
moving an imaging probe to a first location with a robotic arm of a robotic positioning system;
obtaining first image data from the first location, the first image data including information related to at least one anatomical structure;
moving the imaging probe to a second location with the robotic arm;
obtaining second image data from the second location, the second image data including information related to the at least one anatomical structure; and
updating a representation of the at least one anatomical structure based on the first and second image data.

15. The method according to claim 14, further comprising:
moving the imaging probe to a third location with the robotic arm;
obtaining third image data relating to the at least one anatomical structure;
determining a correspondence between the third image data and the 3D anatomical model;
identifying a first discrepancy between the at least one anatomical structure in the third image data and the associated at least one structure in the 3D anatomical model; and
moving the imaging probe with the robotic arm to a fourth location calculated from the first discrepancy.

16. The method of claim 15, further comprising:
obtaining fourth image data relating to the at least one anatomical structure.

17. The method of claim 16, further comprising identifying a second discrepancy between the at least one anatomical structure in the third image data and the at least one anatomical structure in the fourth image data.

18. The method of claim 17, further moving the imaging probe with the robotic arm to a fifth location calculated from the second discrepancy.

19. An imaging system for use in modelling an anatomical structure of a patient, comprising:
a catheter sized and shaped for percutaneous insertion into the patient;
an imaging probe having a field of view, coupled to the catheter near a distal end thereof;
a robotic arm coupled to the catheter and/or the imaging probe, configured to translate and/or rotate the imaging probe;
a mouthpiece configured to be worn by the patient, the mouthpiece being configured to receive the catheter;
a processor operatively coupled to the imaging probe and the drive mechanism, the processor configured to transmit and receive signals with the drive mechanism and the imaging probe, to—
control the robotic arm to place the imaging probe at a first and a second position within the patient, and
control the imaging probe to generate first image data and second image data related to respective first and second fields of view therefrom,
wherein the processor is configured to generate and/or update a model of the anatomical structure considering the first and second image data.

20. The system of claim 19, further comprising one or more sensors disposed on or within the mouthpiece and being configured to measure one of an axial movement of the catheter or a rotation of the catheter with respect to the mouthpiece.

21. The system of claim 19, further comprising a rigid attachment between the mouthpiece and the robotic arm.

22. The system of claim 19, further comprising a rigid attachment between the mouthpiece and a handle of the catheter.

23. The system of claim 19 or 20, wherein the rigid attachment comprises a rack and pinion system.

24. The system of claim 19 or 20, wherein the rigid attachment is configured to prevent excessive forces from being translated by the robotic arm to the patient or the mouthpiece.

25. An imaging system for use in modelling an anatomical structure of a patient, comprising:
a robotically-controlled drive mechanism configured to be coupled to and drive an imaging probe; and
a processor operatively coupled to the imaging probe and the drive mechanism, the processor configured to transmit and receive signals with the drive mechanism and the imaging probe, to—
control the drive mechanism to place the imaging probe at a first and a second position within the patient, and
collect image data from imaging probe at the first and second positions,
wherein the processor is configured to generate and/or update a model of the anatomical structure considering the first and second image data.

26. A robotic system for use in control of an imaging catheter, the robotic system comprising:
a. a base (e.g., comprising lockable wheels) adapted to be repositionable along a floor within an operating room;
b. an arm movably coupled to the base, the arm comprising an interface for receiving a middle portion of an elongate shaft of the imaging catheter;
c. a cradle sized and shaped for receiving an imaging catheter handle therein, the cradle comprising one or more actuators positioned to interface with one or more knobs of the imaging catheter handle when the imaging handle is secured within the cradle, wherein the cradle is adapted for translation and/or rotation; and
d. a controller operatively coupled to the arm and the cradle, the controller programmed to cause movement of the 1) arm, 2) cradle, and/or 3) cradle actuators to adjust a position of the imaging catheter or a configuration of a knob thereof.

27. The robotic system of claim 26 further comprising an imaging console adapted to receive and process imaging data from the imaging catheter.

28. The robotic system of claim 27, wherein the controller is operatively coupled to the imaging console and is further programmed to cause the movement (in d. of claim 1) considering the processed imaging data.

29. A view-based imaging system, comprising:
a catheter;
an imaging element disposed on the catheter;
a console operatively coupled to the catheter and the imaging element, the console being configured to display image information from the catheter and include input controls for selecting a desired view within a patient;
a control system configured to manipulate a position and/or orientation of the catheter and/or imaging element, the control system being configured to move the catheter and/or imaging element within the patient such that the imaging element obtains the desired view; and
one or more processors and memory coupled to the one or more processors, the memory being configured to store computer-program instructions, that, when executed by the one or more processors automatically classifies a present view of the imaging element and provides instructions to the control system to move the imaging element to a location that optimizes the desired view.

30. The system of claim 29, wherein the computer-program instructions are further configured to apply a score to the present view.

31. The system of claim 30, wherein the computer-program instructions are further configured to provide instructions to the control system to move the imaging element until the score for the present view is above a target threshold.

32. The system of claim 30, wherein the computer-program instructions are further configured to provide instructions to the control system to move the imaging element until the score for the present view is maximized.

33. A system comprising:
one or more processors;
memory coupled to the one or more processors, the memory configured to store computer-program instructions, that, when executed by the one or more processors, implement a computer-implemented method, the computer-implemented method comprising:
receiving input controls from a user selecting a desired view of a patient's anatomy;
obtaining a first image from an imaging element of a catheter positioned at a first location within the patient;
applying the first image to a classifier to obtain a score that indicates if the first image corresponds to the desired view;
if the score is above a threshold:
indicating to the user that the first image corresponds to the desired view;
if the score is below the threshold:
providing instructions to move the imaging element of the catheter to a second location; and
applying the second image to the classifier to obtain a new score that indicates if the second image corresponds to the desired view.

34. The system of claim 34, further comprising repeating providing instructions to move the imaging element to subsequent locations and applying images from the subsequent locations until the classifier returns a new score indicating that the image corresponds to the desired view.

35. A method, comprising:
receiving input controls from a user selecting a desired view of a patient's anatomy;
obtaining a first image from an imaging element of a catheter positioned at a first location within the patient;
applying the first image to a classifier to obtain a score that indicates if the first image corresponds to the desired view;
if the score is above a threshold:
indicating to the user that the first image corresponds to the desired view;
if the score is below the threshold:
providing instructions to move the imaging element of the catheter to a second location; and
applying the second image to the classifier to obtain a new score that indicates if the second image corresponds to the desired view.

36. The method of claim 35, further comprising repeating providing instructions to move the imaging element to subsequent locations and applying images from the subsequent locations until the classifier returns a new score indicating that the image corresponds to the desired view.
